(19) United States
(12) Patent Application Publication
(10) Pub. No.: US 2026/0069350 A1
(43) Pub. Date: Mar. 12, 2026

(54) Laparoscopic Applicator, and System for Laparoscopic Ablation

(71) Applicant: Stockert GmbH, Freiburg im Breisgau (DE)

(72) Inventors: Fabian Kees, Freiburg (DE); Patty Ky Ho, Merzhausen (DE); David Stork, Freiburg (DE); Fritz Koch, Freiburg (DE)

(21) Appl. No.: 18/944,629

(22) Filed: Nov. 12, 2024

(30) Foreign Application Priority Data
Sep. 11, 2024 (DE) 102024126105.6

(51) Int. Cl. A61B 18/14; A61B 18/00; A61B 18/12

(52) U.S. Cl. CPC A61B 18/1447; A61B 18/1206; A61B 18/1482; A61B 2018/0016; A61B 2018/00291; A61B 2018/00434; A61B 2018/00577; A61B 2018/00613; A61B 2018/00642; A61B 2018/00791; A61B 2018/00827; A61B 2018/00839; A61B 2018/00875; A61B 2018/00892

(57) ABSTRACT

The present invention describes a device for use in laparoscopic surgery, and a system for laparoscopic ablation. A method for perivascular and/or perineural neuromodulation is also disclosed.

### CROSS-REFERENCE TO RELATED APPLICATIONS

This application claims the benefit of German patent application No. 10 2024 126 105.6 filed on Sep. 11, 2024, the entire disclosures of which are incorporated herein by way of reference.

The present invention is classified in the field of minimally-invasive medical procedures, with special focus on the technique of ablation. It concentrates in particular on the irreversible electroporation (IRE) of tissue, an advanced method which is used in interventional medicine for the targeted treatment of tissue. This technique represents a significant advancement in medical therapy and is already being used in a targeted manner for the ablation of heart tissue, with particular consideration of tissue selectivity, reduced treatment times and the minimisation of risks of conventional treatment methods.

In recent years, the treatment of tissue by means of pulsed electrical fields has become established as an increasingly relevant clinical technique. The use of short high-voltage electrical pulses and the high electrical field strengths associated therewith, which act on the tissue, have, however, already been the subject of intensive research for more than four decades. This application method is categorised as a non-thermal procedure, since it is based on the delivery of short pulses with a high voltage amplitude, which generate between active electrode pairs a locally strong electrical field in the region of up to several thousand volts per centimetre. This field strength leads to the formation for a short time of pores in the cell membranes. If the electrical field exceeds a specific threshold value, which is required for the formation of pores in the lipid bilayers of the cell membranes, and if the tissue is exposed to that field beyond a critical period of time, the cells die as a result of apoptosis.

Pulsed field ablation has already been found to be a highly promising treatment method for the treatment of cardiac arrhythmias, such as, for example, atrial fibrillation. The neuromodulation of nerves by means of pulsed high-voltage electrical signals, on the other hand, is still the subject of numerous research projects. A considerable field of research covers renal denervation. Renal denervation is a method for suppressing increased sympathetic activity in order to treat high blood pressure or other cardiovascular diseases. The current therapeutic approach is a minimally-invasive intervention in which nerve conduction around the renal arteries is prevented with the aid of a special catheter. The catheter is inserted into the renal artery, and the nerve fibres running outside the artery are cauterised through the vessel wall by means of thermal or chemical ablation.

Although the minimally-invasive endovascular catheter-based approach for neuromodulation described above is regarded as a highly promising treatment method, some of the studies which have been carried out give rise to doubts about its effectiveness. Because, for example, the renal sympathetic nerve fibres are located outside the artery and thus cannot be reached directly with a catheter, a laparoscopy-based intervention for perivascular neuromodulation may represent a highly promising alternative procedure. In this manner, the nerve fibres can be brought directly into contact with a laparoscopic ablation instrument and thus the likelihood of successful treatment can be increased.

Irreversible electroporation (IRE) is essentially a non-thermal procedure which uses only a small amount of electrical energy and thus effects an increase in the tissue temperature by only a few ° C. This distinguishes it significantly from conventional RF ablation (RF: radiofrequency), in which the tissue temperature increases by 20 to 70° C. and cells are destroyed by heat. In IRE, bipolar pulses are generally used, that is to say a combination of positive and negative electrical pulses, in order largely to avoid muscle contractions, which usually occur when DC voltage is used. These pulses can be applied between two bipolar electrodes of an applicator or between an applicator electrode and a body surface electrode, which is usually attached to the patient's back.

In order that the IRE pulses generate pores in the tissue, the electrical field strength E, defined by the pulses, at the tissue between a pair of at least two electrodes must exceed a tissue-dependent threshold value Eth. For example, the threshold value for cardiac cells is approximately 500 V/cm, while for bones it is 3000 V/cm. These differences in the threshold values allow the selective application of IRE in different tissues. In order to reach the required field strength, the voltage to applied to an electrode pair depends both on the target tissue and also on the distance between the electrodes and on the size of the electrodes themselves. These parameters likewise influence the thermal energy input during the ablation and thus the temperature peaks which can occur at the tissue to be treated. The applied voltages can reach several kilovolts and are thus significantly above the voltages of 10-200 V that are typical for thermal RF ablation.

Systems for the neuromodulation of nerves and especially for renal denervation that have become established on the market use a transvascular catheter-based approach, either by means of RF energy or ultrasound. However, a general disadvantage of transvascular neuromodulation is that, for cauterisation of the outer nerve fibres, ablation must be carried out through the vessel wall. In order to counteract this, an alternative treatment procedure provides for the use of laparoscopic applicators. These are inserted into the patient's torso and in this manner can address the nerves around the artery in a perivascular manner from outside. The required energy input into the tissue can thus be reduced, as a result of which on the one hand there is the promise of an increased likelihood of successful neuromodulation and at the same time the risk of damage to the vessel can be minimised.

Laparoscopic systems for such perivascular neuromodulation, for example for renal denervation, are known from EP 4335397 A1. That document describes an ablation device including an electrode unit, which is able to enclose a tube-shaped piece of tissue in order to denervate nerves via a thermal ablation procedure (for example radiofrequency ablation).

There is a need for improved ablative approaches.

The present invention relates to laparoscopic ablation, in particular laparoscopy-based neuromodulation.

There are proposed for this purpose a device according to claim 1, a system according to claim 15 and a method according to claim 21.

According to a first aspect, a device for use in laparoscopic surgery is proposed. The device has a shaft. The shaft has a distal end and a proximal end. The device has a holding unit, which is arranged at the distal end of the shaft. The holding unit is adapted to hold and/or to release a tissue. The device has at least two electrodes integrated in/on/with the holding unit. The electrodes can be connected directly and/or permanently to the holding unit. The device has a force transmission unit which is arranged on/in the shaft and is connected, for example indirectly, to the holding unit and which is adapted to be moved relative to the shaft. The device has an electric cable, which is arranged in the shaft and is connected, in particular electrically conductively, to the at least two electrodes and is adapted to transmit an electrical signal that is to be received via the proximal end of the shaft to the at least two electrodes, in particular in order to carry out irreversible electroporation on/in/at a tissue that is being held.

The device can also be referred to as a laparoscopic applicator. The device can have a trocar, which is adapted to insert the shaft through/into a patient's torso.

The device can have an articulated arrangement. The articulated arrangement can be arranged between the holding unit and the distal end of the shaft. The articulated arrangement can be arranged at the distal end of the shaft and/or can be connected, in particular directly, thereto. The articulated arrangement can be adapted to connect the distal end of the shaft, in particular movably, to the holding unit. A movement of the force transmission unit can lead to a movement of the holding unit and/or to the tissue being held and/or released by means of the holding unit. A movement of the force transmission unit can lead to a movement of the articulated arrangement, which in turn leads/can lead to a movement of the holding unit and/or to the tissue being held and/or released.

The holding unit can be configured as a finger gripper, in particular as a two- or three-fingered gripper. The force transmission unit can be configured as a pull- and push-rod. A movement of the pull- and push-rod, in particular relative to the shaft, can lead to the opening or closing of the finger gripper in order to grip/clamp or release the tissue.

The device can have a longitudinal, transverse and vertical axis. The shaft can extend along the longitudinal axis. The pull- and push-rod can be adapted to move along the longitudinal axis. A movement of the pull- and push-rod along the/a, in particular longitudinal, axis leads to an opening movement of the finger gripper along a vertical and/or transverse axis, in particular in the vertical and longitudinal axial plane.

The fingers of the finger gripper can each have an inner face which, in particular when the finger gripper is in a closed state, can be oriented towards the tissue and/or can be in contact with a tissue that is being held. In each case one of the at least two, in particular ablation, electrodes, in particular one of a plurality of electrodes, can be integrated in/on at least one of at least two of the inner faces. At least two electrodes of the plurality of electrodes can be configured as ablation electrodes. At least three of the plurality of electrodes can be configured as measuring electrodes.

The holding unit can be configured as a suction unit. A movement of the force transmission unit can lead to an angling, for example the adjustment of any desired angle, in particular between 0° and 90° inclusive, in particular in a vertical and longitudinal axial plane, of the suction unit relative to the shaft. The suction unit can be adapted to suction a tissue and/or to fix itself to the tissue or to detach itself from the tissue.

The suction unit can have a suction cup. The suction unit can have at least one suction hole, which is arranged in the suction cup. The suction unit can have a suction channel, which is connected to the at least one hole. The suction unit can have a suction connection provided at one end of the suction channel. An external vacuum pump can be connected via the suction connection in order to generate a negative pressure in the suction cup and suction the tissue against/into the suction cup or, by switching off the external vacuum pump or reducing the negative pressure in the suction cup, to detach itself from the tissue.

The suction cup can be configured to be oval, in particular stadium-shaped or circular, along a vertical axis. The at least two, in particular ablation, electrodes can be arranged in the suction cup along the vertical axis and/or transverse to the vertical axis.

The suction cup can have a cup width and a cup length. The suction cup can have a flexible material. For example, the width of the suction cup can thus adapt itself (slightly) as soon as tissue is suctioned. The suction cup can thus adapt itself to a structure and/or shape of the tissue to be suctioned.

The suction cup can be configured to be oval, in particular stadium-shaped or circular, along a vertical axis. At least three, in particular measuring, electrodes can be arranged, for example in addition to the at least two electrodes, in the suction cup along the vertical axis and/or transverse to the vertical axis, in particular spaced apart from the at least two electrodes by a first electrode distance. The at least two/three electrodes can be arranged in the suction cup in an upper/lower region thereof along the vertical axis, while the at least three/two electrodes can be arranged in the suction cup in a lower/upper region thereof transverse to the vertical axis. Formulated in greater detail, the at least two electrodes (e.g. ablation electrodes) can be arranged in the suction cup in an upper region thereof along the vertical axis, while the at least three electrodes (e.g. measuring electrodes) can be arranged in the suction cup in a lower region thereof transverse to the vertical axis. Alternatively, the at least three electrodes (e.g. measuring electrodes) can be arranged in the suction cup in a lower region thereof along the vertical axis, while the at least two electrodes (e.g. ablation electrodes) can be arranged in the suction cup in an upper region thereof transverse to the vertical axis.

The holding unit can be configured as a clamshell gripper, in particular a double half-clamshell gripper. A movement of the force transmission unit can lead to an angling, in particular the adjustment of any desired angle, in particular between 0° and 90° inclusive, in particular in a transverse and longitudinal axial plane, of the clamshell gripper relative to the shaft, and/or to the opening and/or to the closing of the clamshell gripper.

The clamshell gripper can extend and/or have a maximum extent along a transverse axis. The clamshell gripper can be adapted to open or to close along a vertical axis. (The) at least (one of the) two electrodes can be integrated in an inside, in particular at least or solely, of a, for example each, clamshell, in particular can be oriented/extend along and/or transverse to a longitudinal axis. At least two, in particular ablation, electrodes can be integrated in an inside of each clamshell, in particular can be oriented/extend along and/or transverse to a longitudinal axis.

The clamshell gripper can extend and/or have a maximum extent along a transverse axis. The clamshell gripper can be adapted to open or to close along a vertical axis. At least (one of the) three, in particular measuring, electrodes can be integrated, for example in addition to the at least two, in particular ablation, electrodes, in an inside, in particular at least or solely, of one, for example each, clamshell, in particular can be oriented/extend along and/or transverse to a longitudinal axis and/or can be spaced apart from the at least two electrodes by a second electrode distance. At least three, in particular measuring, electrodes can be integrated in an inside of each clamshell, in particular can be oriented/extend along and/or transverse to a longitudinal axis.

The at least two/three electrodes can be arranged in the inside of the clamshell in an upper/lower region along the longitudinal axis, while the at least two/three electrodes can be arranged in a lower/upper region transverse to the longitudinal axis. Or, formulated in greater detail, the at least two electrodes (e.g. ablation electrodes) can be arranged in the inside of the clamshell in an upper region along the longitudinal axis, while the at least three electrodes (e.g. measuring electrodes) can be arranged in a lower region transverse to the longitudinal axis. Alternatively, the at least three electrodes (e.g. measuring electrodes) can be arranged in the inside of the clamshell in a lower region along the longitudinal axis, while the at least two electrodes (e.g. ablation electrodes) can be arranged in an upper region transverse to the longitudinal axis.

The force transmission unit can be configured as a first pull- and push-rod, which is arranged in the shaft and is connected to the articulated arrangement. A movement of the first pull- and push-rod can lead to the opening or closing of the clamshell gripper. The force transmission unit can, in particular additionally, be configured as a second pull- and push-rod, which is arranged in the shaft and is connected, for example solely, to the clamshell gripper. A movement of the second pull- and push-rod can lead to an angling, in particular in a longitudinal-transverse axial plane, for example the adjustment of any desired angle, in particular between 0° and 90° inclusive, of the clamshell gripper relative to the shaft.

The force transmission unit and the holding unit can be configured as a contiguous shape memory material, in particular a shape memory alloy. The holding unit can be configured as a trap structure, in particular a trap basket. A movement of the force transmission unit can lead to the trap structure, in particular the trap basket, moving out of the shaft and unfolding, or to the trap structure, in particular the trap basket, moving back into the shaft and folding.

The trap structure, in particular the trap basket, can have two individual and three contiguous splines. The at least two, in particular ablation, electrodes and/or (in addition to the at least two electrodes) at least three, in particular measuring, electrodes can be integrated in the splines, in particular in the contiguous splines. The three contiguous splines can have approximately the shape of two stadium-shaped splines joined together at their longitudinal sides.

According to a second aspect, a system for laparoscopic ablation, in particular perivascular neuromodulation, is proposed. The system has a device according to the first aspect. The system has a signal generator arrangement which is connected or can be connected to the device. The system has a control and evaluation unit which is connected or can be connected to the device and/or to the signal generator arrangement.

The signal generator arrangement can have a first signal generator for generating a first signal, in particular a radiofrequency (RF) signal, and a second signal generator for generating a second signal, in particular a signal for pulsed field ablation.

This has the advantage that a combination of RF signals and pulsed field ablation is made possible in one system, and the overall procedure of laparoscopic ablation is shortened. Conventionally, two separate systems, which use either RF signals or pulsed field ablation and must alternately be introduced into a patient's thorax, are used for this purpose.

The control and evaluation unit can be adapted to activate the first signal generator and/or the second signal generator to deliver a signal.

The control and evaluation unit can be configured to activate the first signal generator and/or the second signal generator to deliver a signal in dependence on at least one electrode temperature. The control and evaluation unit can be configured to activate the first signal generator to deliver a signal if the electrode temperature falls below a temperature limit value. The control and evaluation unit can be configured to activate the second signal generator to deliver a signal if the electrode temperature assumes or exceeds the temperature limit value.

The control and evaluation unit can be adapted to apply an electric current to the at least two electrodes and/or to measure a voltage via the at least three electrodes, wherein a nerve activity of the tissue and/or the local tissue impedance can be determined from the electric current and the electric voltage.

According to a third aspect, a method for perivascular and/or perineural neuromodulation is proposed. The method comprises inserting an applicator into a patient. The method comprises positioning a distal end of the applicator at a vessel or tissue or nerve of the patient. The method comprises positioning a distal end of the applicator at a vessel or tissue or nerve of the patient such that at least two electrodes of the applicator are in contact perivascularly and/or perineurally with a perimeter of the vessel or tissue or nerve. The method comprises carrying out a denervation by delivering energy via the at least two electrodes. The delivery of energy is effected on the basis of a protocol which has at least one pulse of a pulsed field ablation (PFA). The method comprises withdrawing the applicator.

In the method according to the third aspect an irreversible electroporation may be carried out.

In the method according to the third aspect a reversible electroporation may be carried out.

The distal end of the applicator can alternatively be positioned at a tissue, correspondingly to the positioning at a vessel or tissue or nerve. A generator system can be provided for carrying out the denervation. The generator can be electrically connected to at least two electrodes integrated or provided distally at the applicator / at the distal end of the applicator.

The applicator can be inserted by means of a laparoscopic procedure. In addition or alternatively, the applicator can be withdrawn by means of a laparoscopic procedure.

The laparoscopic applicator can be introduced into a patient's torso by means of a trocar. The applicator can have the trocar. The applicator can further comprise a shaft having a distal end and a proximal end. The distal end can be equipped, for example, with a plurality of electrodes.

The applicator can be inserted by means of an open surgical procedure. In addition or alternatively, the applicator can be withdrawn by means of an open surgical procedure.

After the applicator has been inserted, geometric unfolding of a distal end of the applicator can be initiated. A form of the distal end that conforms to a vessel or tissue or nerve can thus be achieved. Unfolding can be effected by a mechanical, magnetic or material-based controller.

The electrodes of the applicator can be brought into contact with a (target) vessel or tissue or nerve.

The method can further comprise checking a positioning of the applicator by means of a local impedance measurement. For example, positioning of the applicator can be regarded as complete when at least two electrodes are in contact with the vessel or tissue or nerve. The positioning can subsequently be checked.

The protocol can comprise, for example, only pfa pulses.

The protocol can comprise a combination of PFA pulses and radiofrequency (RF) pulses.

The method can further comprise choosing whether the delivery of energy for the denervation is to be carried out on the basis of a protocol by means of PFA pulses or on the basis of a protocol by means of a combination of PFA pulses and RF pulses.

A delivery of energy can be effected once or multiple times at the same or different locations of the vessel or tissue or nerve. The delivery of energy can be effected by means of PFA pulses or by means of a combination of PFA pulses and RF pulses.

In the method according to the third aspect, perivascular and/or perineural neuromodulation occurs by carrying out the denervation. Neuromodulation through a vessel wall is thus not necessary and can be avoided.

The method can further comprise, before the denervation is carried out, quantifying a conductivity of the vessel or tissue or nerve by means of at least two stimulation and measuring electrodes. The quantification of the conductivity can be effected via a stimulation and measuring device, which can have stimulation and measuring electrodes arranged distally and proximally to at least one ablation electrode.

The method can further comprise, after the denervation has been carried out, characterising the denervation by means of at least two stimulation and measuring electrodes. The characterisation of the denervation can be effected via a stimulation and measuring device, which can have stimulation and measuring electrodes arranged distally and proximally to at least one ablation electrode.

While the described procedure is being carried out, a temperature at an ablation site chosen for the ablation can be measured. In other words, the method can further comprise measuring the temperature at the chosen ablation site while the procedure is being carried out.

Further features, properties, advantages and possible modifications will become clear to a person skilled in the art from the following descriptions, in which reference is made to the accompanying drawings.

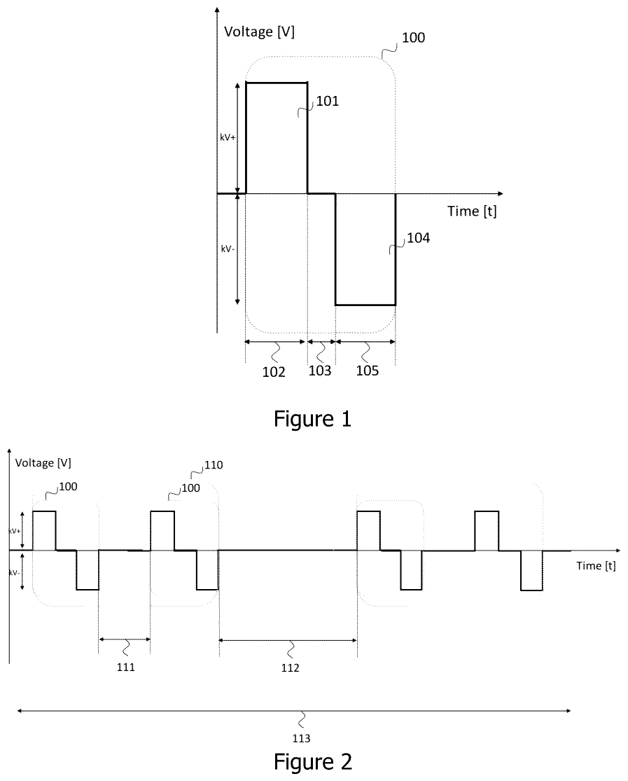

FIG. 1 is a schematic representation of a biphasic ire pulse according to a variant of an embodiment.

FIG. 2 is a schematic representation of a pulse protocol with multiple bursts of biphasic pulses according to a variant of an embodiment.

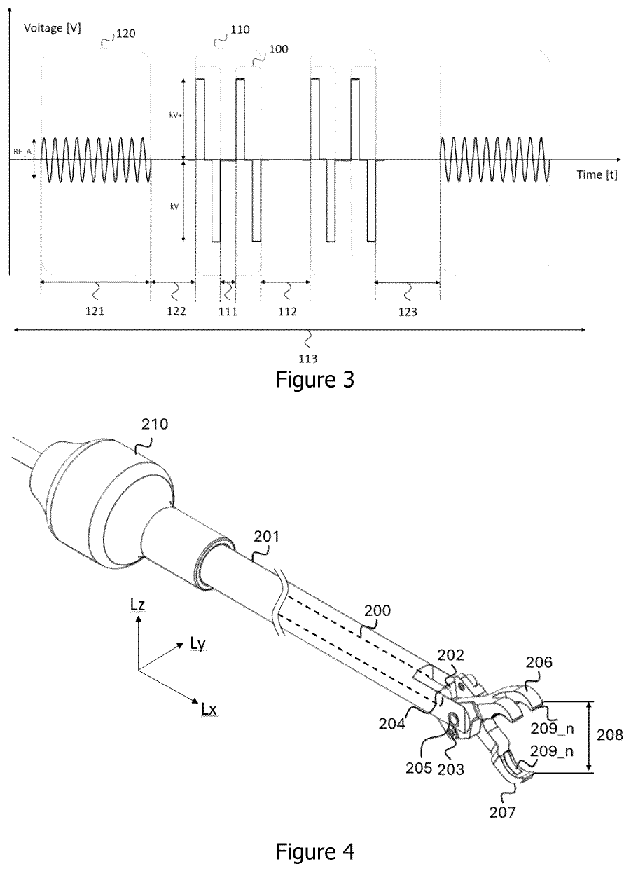

FIG. 3 shows a schematic representation of a procedure protocol, with at least one burst of biphasic IRE pulses combined with at least one RF energy burst 120, according to a variant of an embodiment.

FIG. 4 Shows an Embodiment of the Distal Portion of a Laparoscopic applicator.

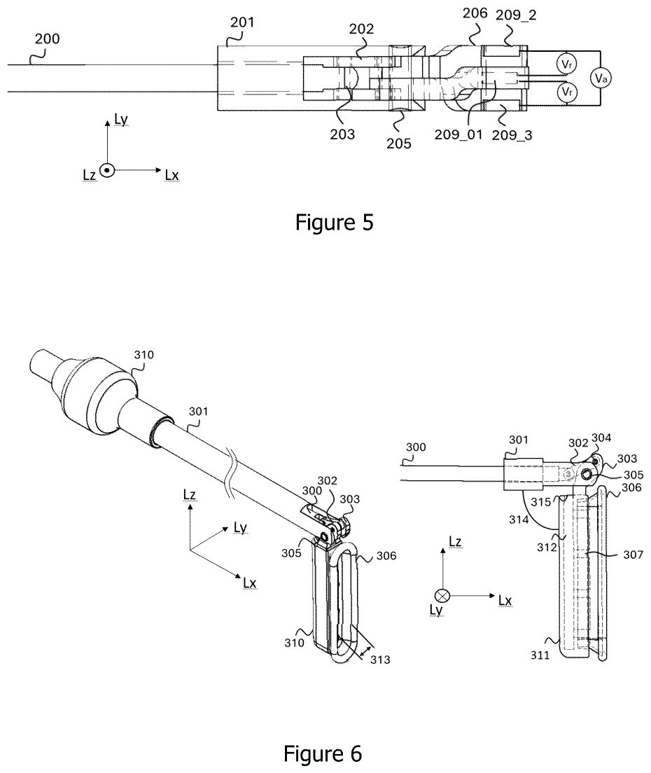

FIG. 5 shows schematic representations for the activation of the above-described electrodes to carry out a neuromodulation procedure.

FIG. 6 Shows an Embodiment of the Distal Portion of a Laparoscopic applicator.

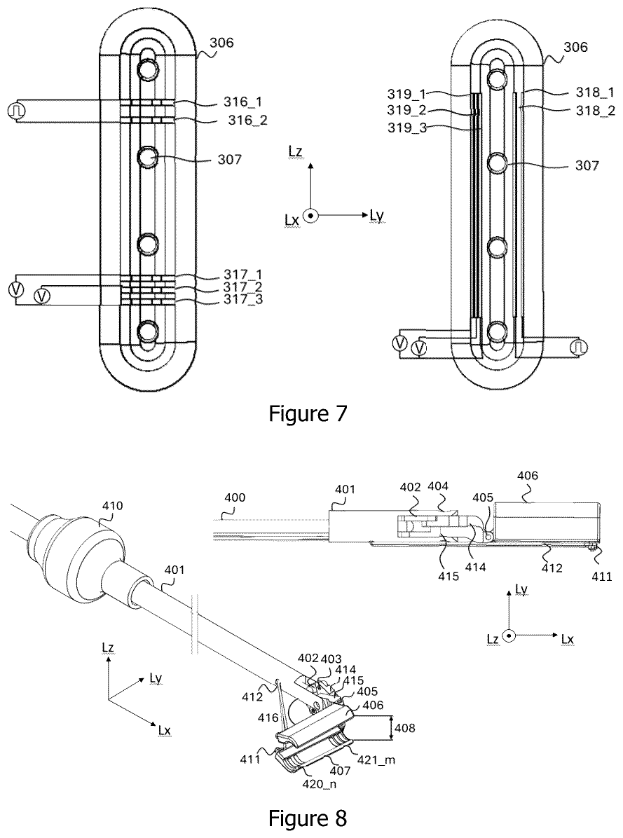

FIG. 7 shows schematic representations of electrode arrangements in the interior of a suction cup and activation possibilities for carrying out a procedure of neuromodulation.

FIG. 8 shows an embodiment of the distal portion of a laparoscopic applicator.

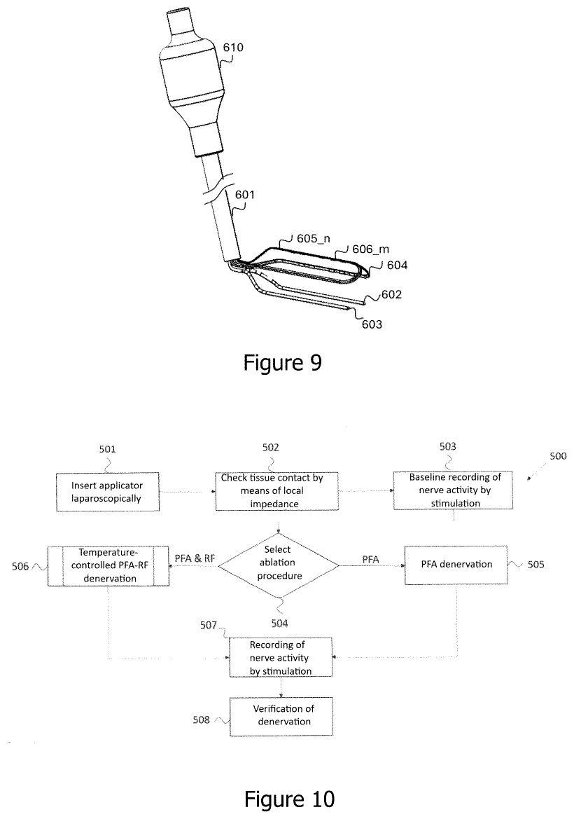

FIG. 9 shows a further embodiment of the distal portion of a laparoscopic applicator.

FIG. 10 shows a schematic sequence for carrying out laparoscopic neuromodulation using the example of a denervation procedure.

FIG. 1 is a schematic representation of a biphasic IRE pulse (irreversible electroporation) according to an embodiment variant. It shows the voltage V of the biphasic PFA pulse 100 as a function of the time t in an IRE ablation procedure. The present embodiment variant relates to a second signal generator in the form of an IRE generator, which is configured as a voltage source. Consequently, the IRE signals are here described in the form of their voltages. The biphasic IRE pulse 100 comprises a positive pulse 101 and a negative pulse 104, wherein the terms “positive” and “negative” refer to an independently chosen polarity of two electrodes which are activated for the ablation and between which the biphasic pulse is applied. The amplitude of the positive pulse 101 is denoted kV+ and lasts for time 102. Analogously, the amplitude of the negative pulse 104 is denoted kV− and has a duration 105. Between the two pulse phases 101 and 104 there is a delay time 103. Both the two temporal pulse widths 102 and 105 and the amplitudes kV+ and kV− can be configured independently of one another and can therefore vary in an exemplary embodiment of the invention.

FIG. 2 is a schematic representation of a pulse protocol with multiple bursts of biphasic pulses according to an embodiment variant. Over the duration of the entire IRE procedure 113, the pulses 100 are delivered in the form of one or more bursts or pulse packets 110. Each burst 110 comprises a defined number N of biphasic pulses 100, wherein the pulses are separated by a time interval 111. There is again a delay time 112 between the delivery of the individual bursts 110.

FIG. 3 shows a schematic representation of a procedure protocol with at least one burst of biphasic IRE pulses combined with at least one RF energy burst 120 according to an embodiment variant. Over the duration of the entire combined procedure 113, the RF energy and the IRE pulses are delivered in the form of one or more bursts 120 and 110. Each IRE burst comprises a defined number N of bipolar pulses 100, wherein the pulses are separated by a time interval 111. The RF burst is described by a sinusoidal signal of amplitude RF_A and duration 121. Following an RF burst there is a delay time 122. Both the duration of the RF burst and the subsequent delay time 122 can be adjusted on the basis of the instantaneously measured temperature at the ablation electrodes. There is a delay time 112 between the delivery of the individual IRE bursts 110. There is a delay time 123 between an IRE burst and delivery of a further RF burst.

FIG. 4 shows an embodiment variant of the distal portion of a laparoscopic applicator. The applicator has a shaft 201, which can be inserted through a patient's torso with the aid of a trocar 210. In the interior of the shaft 201 there extends a pull- and push-rod 200, which is movable freely and relative to the shaft along a longitudinal axis Lx. At the distal end of the applicator there is a three-fingered gripper 206, 207 having a two-fingered part 206 and a one-fingered part 207. Each of the three fingers has an inner side, in particular a concave indentation. The three-fingered gripper 206, 207 is adapted to grip a cylindrical tissue structure, the circumference of which is formed about a transverse axis Ly. The cylindrical tissue structure has a longitudinal extent which is oriented parallel to the transverse axis Ly of the laparoscopic applicator. In order to implement the gripping mechanism, the three-fingered gripper 206, 207 is mounted via an articulated arrangement 202, 203, 204, 205 to the outer shaft 201 and also to the inner pull- and push-rod 200 such that a movement of the pull- and push-rod 200 along the longitudinal axis Lx leads to gripping of the three-finger gripper. A gripping distance 208 is thus variably adjustable and accordingly adaptable to different tissue structures. A plurality of electrodes 209-n are integrated in the concave indentations of the three-fingered gripper 206, 207. Each concave indentation has one electrode. Each of the electrodes 209-n is connected via electrical contacts, which extend in the interior of the shaft 201, to an evaluation and control unit (not shown) as well as to the generator system (not shown). These electrodes are each exposed to an external environment, whereas the electrical connections to the control unit and the generator system are insulated with respect to the external environment. In the present embodiment variant, the electrodes are configured as ablation electrodes, whereby laparoscopic ablation is made possible.

FIG. 5 shows schematic representations for activation of the above-described electrodes 209-n to carry out a neuromodulation procedure. In this embodiment, the electrodes 209_2 and 209_3 are located on the two-fingered part 206 and electrode 209_1 is located on the one-fingered part 207. Activation possibility 1 provides for the generation of an electrical field in the radial direction. To this end, the two voltages Vr are applied between the electrode pair 209_2 and 209_1 as well as between the electrode pair 209_3 and 209_1. When all the electrodes are in contact with a tissue structure, a radially oriented current flow is obtained. In the case of activation possibility 2, a current flow oriented along the transverse axis Ly is induced by application of an electrical field by means of a voltage Va between the electrode pair 209_2 and 209_3. Electrode 209_1 does not have a function here and assumes an electrically floating state.

FIG. 6 shows a further embodiment variant of the distal portion of a laparoscopic applicator. The applicator has an outer shaft 301, which can be inserted through a patient's torso with the aid of a trocar 310. In the interior of the shaft 301 there extends a pull- and push-rod 300, which is movable freely and relative to the shaft 301 along a longitudinal axis Lx. At the distal end of the applicator there is a suction unit 311 having a flexible atraumatic suction cup 306, which is able to establish contact with a cylindrical tissue structure by means of negative pressure. The suction cup 311 is connected via an articulated arrangement 302, 303, 304, 305 to the shaft 301 and to the pull- and push-rod 300 such that an angle 314 between the longitudinal axis Lx of the shaft 301 and the suction unit 311 can freely be adjusted. With the aid of the adjustable angle, both insertion through the trocar 310 can be achieved and optimal contact with the tissue structure can be made possible. In the interior of the suction cup 306 there is a plurality of suction holes 307, which are connected to a suction channel 312. The suction cup has a cup width 313. At the outlet of the suction channel 312 there is a vacuum connection 315, which establishes a connection to an external vacuum pump in order to generate a negative pressure in the interior of the suction cup 306.

FIG. 7 shows schematic representations of electrode arrangements in the interior of the suction cup 306 and activation possibilities for carrying out a procedure of neuromodulation. In electrode arrangement 1 (on the left) there are two ablation and stimulation electrodes 316_1 and 316_2, as well as three measuring electrodes 371_1, 317_2 and 317_3. All the electrodes are arranged such that they are oriented perpendicular to a longitudinal axis of a suctioned cylindrical tissue structure. Via the two electrodes 316_1 and 316_2, a baseline stimulation of the nerves can be carried out at the start of the procedure by application of a defined electric current to that electrode pair. With the aid of the three measuring electrodes 371_1, 317_2 and 317_3, the stimulus conduction of the nerves is measured while they are simultaneously being stimulated. To that end, two bipolar voltages are detected via the electrode pair 317_1 and 317_2 and via the electrode pair 371_2 and 317_3 and are processed. Following this baseline measurement, bipolar ablation is effected via electrode pair 316_1 and 316_2. In order to characterise the neuromodulation, the nerve conductivity is again measured and analysed, as described above, following the ablation. The sequence of this procedure is identical in electrode arrangement 2 (on the right). However, it differs in the arrangement of the electrodes within the suction cup 306. In this configuration, all the electrodes are oriented parallel to a longitudinal axis of a suctioned cylindrical tissue structure. The two ablation and stimulation electrodes 318_1 and 318_2 are located on the right of the plurality of suction holes 307, and the measuring electrodes 319_1, 319_2 and 319_3 are located on the left of the plurality of suction holes 307.

FIG. 8 shows an embodiment variant of the distal portion of a laparoscopic applicator. It has an outer shaft 401, which can be inserted through a patient's torso with the aid of a trocar 410. In the interior of the shaft 401 there extends a first pull- and push-rod 400, which is freely movable along a longitudinal axis Lx. At the distal end of the applicator there is a double half-clamshell gripper having a first half-clamshell 406 and a second half-clamshell 407, which each have a concave indentation and are adapted to be able to grip a cylindrical tissue structure. In order to implement the gripping mechanism, the double half-clamshell gripper is mounted via an articulated arrangement 402, 403, 404, 405, 414, 415 to the outer shaft 401 as well as to the inner pull- and push-rod 400, such that a movement of the pull- and push-rod 400 along the longitudinal axis Lx leads to clamping of the first half-clamshell 406 and of the second half-clamshell 407. A clamping distance 408 that is achieved is thus variably adjustable and adaptable to different tissue structures. In the interior of the shaft 401 there extends a second pull- and push-rod 412, which is connected to the first half-clamshell 406 and to the second half-clamshell 407 such that an angle 416 between the longitudinal axis Lx of the shaft 401 and the double half-clamshell gripper can be adjusted. With the aid of the adjustable angle 416, both insertion through the trocar 410 can be achieved and optimal contact with the tissue structure can be made possible. On the concave indentations of the first half-clamshell 406 and of the second half-clamshell 407 there are a plurality of ablation and stimulation electrodes 420-n as well as measuring electrodes 421_m, which are individually connected via electrical contacts, which extend in the interior of the shaft 401, to the evaluation and control unit as well as to the generator system (both not shown). These electrodes are each exposed to the external environment, while the electrical connections to the control unit and to the generator system are insulated from the external environment. Using the example of two ablation and stimulation electrodes and three measuring electrodes, activation is effected analogously to the description in FIG. 7.

FIG. 9 shows a further embodiment variant of the distal portion of a laparoscopic applicator. The applicator has an outer shaft 601, which can be inserted through a patient's torso with the aid of a trocar 610. In the interior of the shaft 601 there extends a trap structure, for example a trap basket, for example made of a shape memory material, which is composed of three contiguous splines 604 and two individual splines 602 and 603. The entire trap structure is resilient, such that it can be pulled completely into the interior of the shaft 601. It is adapted to be able to grip and clasp, in the state shown, a cylindrical tissue structure. On the three contiguous splines 604 there are in each case a plurality of stimulation and ablation electrodes 605-n as well as a plurality of measuring electrodes 606_m. Each of the plurality of electrodes 605-n, 606_m is individually connected via electrical contacts, which extend in the interior of the shaft 601, to the evaluation and control unit as well as to the generator system (both not shown). The electrodes are each exposed to the external environment, while the electrical connections to the evaluation and control unit and to the generator system are insulated from the external environment. Using the example of two ablation and stimulation electrodes and three measuring electrodes, activation is effected analogously to the description in FIG. 7.

FIG. 10 shows a schematic sequence 500 for carrying out a laparoscopic neuromodulation using the example of a denervation procedure. One or more of the steps described in relation to FIG. 10 can be carried out using a laparoscopic applicator as has been described by way of example in relation to FIGS. 4, 6, 8 and 9. In the first step 501, an applicator system, in particular a laparoscopic applicator, is inserted laparoscopically into the patient. As described by way of example in relation to FIGS. 4, 6, 8 and 10, the laparoscopic applicator can have a trocar by means of which a shaft of the laparoscopic applicator can be inserted into a patient's torso. After the applicator has been positioned, it is checked, by measurement of the local impedance at the ablation electrodes, whether the electrodes have sufficiently good contact with the tissue (step 502). In the next step 503, the starting value of the nerve conductivity is recorded, for example by the mechanisms described above in relation to FIG. 7. For example, by applying a defined electric current to an electrode that is distal or proximal to an ablation electrode, a stimulation pulse can be delivered to one or more nerves. A resulting bipolar voltage is measured at measuring electrode pairs while the nerve is simultaneously being stimulated, and is used to characterise the nerve conductivity. Then, in step 504, it is possible to choose between two types of procedure or input options: denervation by means of pulsed field ablation (PFA) or a combination procedure of PFA and RF energy. In the case of PFA denervation (step 505), the procedure, in particular a delivery of energy, is effected on the basis of an exemplary protocol as described in FIG. 2 by means of biphasic IRE pulses. If a PFA-RF combination procedure (506) is chosen, the procedure, in particular a delivery of energy, is effected on the basis of a combination protocol as described by way of example in FIG. 3. Independently of the choice of the denervation procedure, the nerve activity is then recorded again (507) in order to characterise the denervation (508) as described in FIG. 7, FIG. 8 and FIG. 9.

In addition to an advantageous applicator, a laparoscopy-based neuromodulation of nerves by a combined procedure consisting of successive IRE and radiofrequency (RF) sequences is described herein. In this manner, the tissue conductivity can be increased in a targeted manner while the temperature is monitored continuously. The success of the irreversible electroporation can thus be maximised and at the same time the occurrence of potentially damaging high current densities can be reduced. By using the applicator, ablation does not have to be carried out through the vessel wall.

1. Device for use in laparoscopic surgery, having
a shaft, which has a distal end and a proximal end,
a holding unit, which is arranged at the distal end of the shaft and is adapted to hold and/or to release a tissue,
at least two electrodes integrated in the holding unit,
a force transmission unit, which is arranged on the shaft, is connected to the holding unit and is adapted to be moved relative to the shaft,
an electric cable, which is arranged in the shaft, is connected to the at least two electrodes and is adapted to transmit an electrical signal that is to be received via the proximal end of the shaft to the at least two electrodes.

2. Device according to claim 1, having an articulated arrangement, which is arranged between the holding unit and the distal end of the shaft and is adapted to connect the distal end of the shaft, in particular movably, to the holding unit, wherein a movement of the force transmission unit leads to a movement of the holding unit and/or to the tissue being held and/or released by means of the holding unit.

3. Device according to claim 1, wherein the holding unit is configured as a finger gripper, in particular a three-fingered gripper, and the force transmission unit is configured as a pull- and push-rod, wherein a movement of the pull- and push-rod leads to the opening or closing of the finger gripper in order to grip or release the tissue.

4. Device according to claim 3, wherein fingers of the finger gripper each have an inner face oriented, in particular when the finger gripper is in a closed state, towards the tissue, in which inner face there is integrated at least one of the at least two electrodes, in particular one of a plurality of electrodes.

5. Device according to claim 1, wherein the holding unit is configured as a suction unit, wherein a movement of the force transmission unit leads to an angling of the suction unit relative to the shaft, and/or the suction unit is adapted to suction a tissue or to detach itself from the tissue.

6. Device according to claim 5, wherein the suction unit has:
a suction cup,
at least one suction hole, which is arranged in the suction cup,
a suction channel, which is connected to the at least one suction hole, and/or
a suction connection provided at the end of the suction channel, via which an external vacuum pump can be connected in order to generate a negative pressure in the suction cup and suction the tissue against the suction cup.

7. Device according to claim 6, wherein the suction cup is configured to be oval, in particular stadium-shaped or circular, along a vertical axis, and the at least two electrodes are arranged in the suction cup along the vertical axis and/or transverse to the vertical axis.

8. Device according to claim 6, wherein the suction cup is configured to be oval, in particular stadium-shaped or circular, along a vertical axis, and at least three electrodes are arranged in the suction cup along the vertical axis and/or transverse to the vertical axis, in particular spaced apart from the at least two electrodes.

9. Device according to claim 1, wherein the holding unit is configured as a clamshell gripper, in particular a double half-clamshell gripper, wherein a movement of the force transmission unit leads to an angling of the clamshell gripper relative to the shaft, and/or to the opening and/or to the closing of the clamshell gripper.

10. Device according to claim 9, wherein the clamshell gripper extends along a transverse axis and is adapted to open or to close along a vertical axis, wherein the at least two electrodes are integrated in an inside of at least one clamshell, in particular along a longitudinal axis.

11. Device according to claim 9, wherein the clamshell gripper extends along a transverse axis and is adapted to open or to close along a vertical axis, wherein at least three electrodes are integrated in an inside of at least one clamshell, in particular along and/or transverse to a longitudinal axis, spaced apart from the at least two electrodes.

12. Device according to claim 9, wherein the force transmission unit is configured as a first pull- and push-rod, which is arranged in the shaft and is connected to the articulated arrangement, wherein a movement of the first pull- and push-rod leads to the opening or closing of the clamshell gripper, and/or the force transmission unit is configured as a second pull- and push-rod, which is arranged in the shaft and is connected, in particular solely, to the clamshell gripper, wherein a movement of the second pull- and push-rod leads to an angling of the clamshell gripper relative to the shaft.

13. Device according to claim 1, wherein the force transmission unit and the holding unit are configured as a contiguous shape memory material, in particular shape memory alloy, and the holding unit forms a trap structure, in particular a trap basket, wherein a movement of the force transmission unit leads to the trap structure, in particular the trap basket, moving back into the shaft and folding, or leads to the trap structure, in particular the trap basket, moving out of the shaft and unfolding.

14. Device according to claim 13, wherein the trap structure has two individual and three contiguous splines, wherein the at least two electrodes and/or at least three electrodes are integrated in the splines, in particular in the contiguous splines.

15. System for laparoscopic ablation, having:
a device according to claim 1;
a signal generator arrangement which is connected or can be connected to the device; and
a control and evaluation unit which is connected or can be connected to the device and/or to the signal generator arrangement.

16. System according to claim 15, wherein the signal generator arrangement has a first signal generator for generating a radiofrequency, RF, signal and a second signal generator for generating a signal for pulsed field ablation.

17. System according to claim 15, wherein the control and evaluation unit is configured to activate the first signal generator and/or the second signal generator to deliver a signal.

18. System according to claim 17, wherein the control and evaluation unit is configured to activate the first signal generator and/or the second signal generator to deliver a signal in dependence on at least one electrode temperature.

19. System according to claim 18, wherein the control and evaluation unit is configured to activate the first signal generator to deliver a signal if the electrode temperature falls below a temperature limit value and to activate the second signal generator to deliver a signal if the electrode temperature assumes or exceeds the temperature limit value.

20. System according to claim 15, wherein the control and evaluation unit is adapted to apply an electric current to the at least two electrodes and/or to measure a voltage via the at least three electrodes, wherein a nerve activity of the tissue and/or the local tissue impedance can be determined or is determined from the electric current and the electric voltage.

21. Method for perivascular and/or perineural neuromodulation, comprising:
inserting an applicator into a patient;
positioning a distal end of the applicator at a vessel or tissue or nerve of the patient such that at least two electrodes of the applicator are in contact perivascularly and/or perineurally with a perimeter of the vessel or tissue or nerve or lie against a perimeter of the vessel or tissue or nerve;
carrying out a denervation by delivering energy via the at least two electrodes, wherein the delivery of energy is effected on the basis of a protocol which has at least one pulse of a pulsed field ablation (PFA);
withdrawing the applicator.

22. Method according to claim 21, wherein an irreversible electroporation is carried out.

23. Method according to claim 21, wherein a reversible electroporation is carried out.

24. Method according to claim 21, wherein the applicator is inserted and/or withdrawn by a laparoscopic procedure.

25. Method according to claim 22, wherein the laparoscopic applicator is inserted into a patient's torso by means of a trocar.

26. Method according to claim 21, wherein the applicator is inserted and/or withdrawn by means of an open surgical procedure.

27. Method according to claim 21, wherein, after the applicator has been inserted, geometric unfolding of a distal end of the applicator is initiated and a form of the distal end that conforms to a vessel or tissue or nerve is thus achieved.

28. Method according to claim 21, wherein the electrodes of the applicator are brought into contact with a target vessel or target tissue or target nerve.

29. Method according to claim 21, further comprising:
checking a positioning of the applicator by means of a local impedance measurement.

30. Method according to claim 21, wherein the protocol comprises only PFA pulses.

31. Method according to claim 21, wherein the protocol comprises a combination of PFA pulses and radiofrequency (RF) pulses.

32. Method according to claim 21, further comprising:
choosing whether the delivery of energy for the denervation is to be carried out on the basis of a protocol by means of PFA pulses or on the basis of a protocol by means of a combination of PFA pulses and RF pulses.

33. Method according to claim 21, wherein a delivery of energy is effected once or multiple times at the same or different locations of the vessel or tissue or nerve.

34. Method according to claim 21, wherein perivascular and/or perineural neuromodulation occurs by carrying out the denervation.

35. Method according to claim 21, further comprising:
before the denervation is carried out, quantifying a conductivity of the vessel or tissue or nerve by means of at least two stimulation and measuring electrodes.

36. Method according to claim 21, further comprising:
after the denervation has been carried out, characterising the denervation by means of at least two stimulation and measuring electrodes.

37. Method according to claim 21, further comprising:
measuring a temperature at a chosen ablation site.
